(19) United States
(12) Patent Application Publication
(10) Pub. No.: US 2008/0286352 A1
(43) Pub. Date: Nov. 20, 2008

(54) Liposome Compositions

(76) Inventors: Saran Kumar, Edison, NJ (US); Wenlei Jiang, Bridgewater, NJ (US); Jorg Ogorka, Steinen (DE); Jia-ai Zhang, Skillman, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/065,134

(22) PCT Filed: Aug. 31, 2006
(86) PCT No.: PCT/US06/34234

§ 371 Date: Feb. 28, 2008

Related U.S. Application Data
(60) Provisional application No. 60/713,273, filed on Sep. 1, 2005.

(51) Int. Cl. A61K 9/127; A61K 31/66; A61P 43/00

(52) U.S. Cl. 424/450; 514/109

(57) ABSTRACT

A method of liposome-based therapy for a mammalian subject is disclosed. The method uses liposomes and/or liposomes with outer surfaces that contain an affinity moiety effective to bind specifically to a biological surface at which the therapy is aimed, and a hydrophilic polymer coating. The hydrophilic polymer coating is made up of polymer chains covalently linked to surface lipid components. After a desired liposome biodistribution is achieved, the affinity agent binds to the target surface and helps internalize the liposomes.

### FIELD OF THE INVENTION

The present invention relates to a therapeutic composition and method that employs, as the delivery vehicle, liposomes having a divalent cation matrix. The divalent cation matrix shields the therapeutic agent. The liposomes optionally comprise of an affinity moiety on the outer liposome surfaces for effective binding and internalization by target tissues. The liposomes optionally also comprise a surface coating of hydrophilic polymers for steric stability and prolonged circulation.

### BACKGROUND OF THE INVENTION

Liposomes are used for a variety of therapeutic purposes, in particular, for carrying therapeutic agents to target cells by systemic administration of liposomes.

For a variety of reasons, it may be desirable to shield a therapeutic agent using a liposome. In order to exploit the therapeutic effects of the bisphosphonate class of drugs, the drug distribution must be altered in a way so the therapeutic agent can effectively interact specifically to a target surface at which the therapy is aimed. Therefore, it is desirable to provide a therapeutic liposome composition including a divalent cation matrix where the therapeutic agent is shielded.

### SUMMARY OF THE INVENTION

In one aspect, the invention includes a method of liposome-based therapy for a mammalian subject which includes systemically administering to the subject, liposomes containing (i) a divalent cation matrix effective and (ii) a therapeutic agent. The divalent cation matrix provides protection of a therapeutic agent which otherwise might leak out of traditional liposomal formulation once introduced into the body.

Another aspect, the invention includes a method of liposome-based therapy for a mammaliam subject which includes systemically administering to the subject liposomes containing (i) a divalent cation matrix, (ii) a therapeutic agent, (iii) a hydrophilic polymer coating for steric stability and prolonged circulation; and (iv) optionally an affinity moiety effective to bind specifically to a target surface at which the therapy is aimed The hydrophilic polymer coating is made up of polymer chains which are covalently linked to surface lipid components in the liposomes.

In one embodiment the divalent cation matrix contains divalent cations, such as calcium ions, zinc ions, magnesium ions.

In one embodiment, where a therapeutic agent is to be administered to a target region, the affinity moiety is a ligand effective to bind specifically with a receptor at the target region, and the liposomes include the therapeutic agent in entrapped form. An example of this embodiment is treatment of a solid tumor, where the affinity moiety is effective to bind specifically to a tumor-specific antigen, the liposomes have an average size between about 10 to about 500 nm and include an entrapped drug.

In one embodiment the divalent cation matrix contains cationic lipid. Such lipid is this containing a sterol, an acyl or diacyl chain, where the lipid has an overall net positive charge. Exemplary lipids include 1,2-diacyl-3-trimethylammonium-propane (DOTAP), dimethyldioctadecylammonium (DDAB), N-[1-(2,3,-ditetradecyloxy) propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3β[N—(N′,N′-dimethylaminoethane) carbamoly] cholesterol (DC-Chol);.

### DETAILED DESCRIPTION OF THE INVENTION

### I. Liposome Composition

A liposome for use in liposome-based therapy, has at least one outer bilayer having an outer surface. It will be appreciated that the liposome may include additional bilayers. The outer bilayer is composed of interior and exterior lipid layers, respectively, of the bilayer, each layer being composed of vesicle-forming lipids, such as phospholipids and cholesterol, typically having a diacyl hydrophobic lipid tail and a polar head group. Liposome is composed primarily of such vesicle-forming lipids.

The liposome comprises divalent cations to effectively shield the therapeutic agent from leaching out before it is exposed for interaction with its target. The divalent cation matrix decreases the permeability of the therapeutic agent across the liposome bilayers by trapping the drug. A divalent cation matrix assists in trapping therapeutic agents that are highly soluble. In addition, a divalent cation matrix can facilitate therapeutic agents delivery to tumor more efficiently.

In one embodiment, calcium ions incorporated into the liposome helps to retain the active drug from dispersing before reacting the target.

A therapeutic agent to be administered to a target cell or region is entrapped in a liposome. As used herein, therapeutic agent, compound and drug are used interchangeably. The therapeutic agent may be entrapped in the inner aqueous compartment of the liposome or in the lipid bilayer, depending on the nature of the compound.

The entrapped therapeutic agent may be any of a large number of therapeutic agents that can be entrapped in lipid vesicles, including water-soluble agents that can be stably encapsulated in the aqueous compartment of the vesicles, lipophilic compounds that stably partition in the lipid phase of the vesicles, or agents that can be stably attached, e.g., by electrostatic attachment to the outer vesicle surfaces. Exemplary water-soluble compounds include the bisphosphonate class of drugs. Examples of a therapeutic agent are substituted alkanediphosphonic acids, in particular to heteroarylalkanediphosphonic acids of formula I

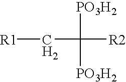

wherein R1 is a 5-membered heteroaryl radical which contains, as hetero atoms, 2 to 4 N-atoms or 1 or 2 N-atoms as well as 1 O- or S-atom, and which is unsubstituted or C-substituted by lower alkyl, phenyl or phenyl which is substituted by lower alkyl, lower alkoxy and/or halogen, or by lower alkoxy, hydroxy, di-lower alkylamino, lower alkylthio and/or halogen, and/or is N-substituted at a N-atom which is capable of substitution by lower alkyl, lower alkoxy and/or halogen, and R2 is hydrogen, hydroxy, amino, lower alkylthio or halogen, and to the salts thereof, to the preparation of said compounds, to pharmaceutical compositions containing them, and to the use thereof as medicaments.

Examples of 5-membered heteroaryl radicals containing 2 to 4 N-atoms or 1 or 2 N-atoms as well as 1 O- or S-atom as hetero atoms are: imidazolyl, e.g. imidazol-1-yl, imidazol-2-yl or imidazol-4-yl, pyrazolyl, e.g. pyrazol-1-yl or pyrazol-3-yl, thiazolyl, e.g. thiazol-2-yl or thiazol-4-yl, or, less preferably, oxazolyl, e.g. oxazol-2-yl or oxazol-4-yl, isoxazolyl, e.g. isooxazol-3-yl or isooxazol-4-yl, triazolyl, e.g. 1H-1,2,4-triazol-1-yl, 4H-1,2,4-triazol-3-yl or 4H-1,2,4-triazol-4-yl or 2H-1,2,3-triazol-4-yl, tetrazolyl, e.g. tetrazol-5-yl, thiadiazolyl, e.g. 1,2,5-thiadazol-3-yl, and oxdiazolyl, e.g. 1,3,4-oxadiazol-2-yl. These radicals may contain one or more identical or different, preferably one or two identical or different, substituents selected from the group mentioned at the outset. Radicals R1, unsubstituted or substituted as indicated, are e.g. imidazol-2-yl or imidazol-4-yl radicals which are unsubstituted or C-substituted by phenyl or phenyl which is substituted as indicated, or which are C- or N-substituted by C₁-C₄ alkyl, e.g. methyl, and are typically imidazol-2-yl, 1-C₁-C₄ alkylimidazol-2-yl such as 1-methylimidazol-2-yl, or 2- or 5-C₁-C₄ alkylimidazol-4-yl such as 2- or 5-methylimidazol-4-yl, unsubstituted thiazolyl radicals, e.g. thiazol-2-yl, or 1H-1,2,4-triazol radicals, unsubstituted or substituted by C₁-C₄ alkyl such as methyl, e.g. 1-C₁-C₄ alkyl-1H-1,2,4-triazol-5-yl such as 1-methyl-1H-1,2,4-triazol-5-yl, or imidazol-1-yl, pyrazolyl-1-yl, 1H-1,2,4-triazol-1-yl, 4H-1,2,4-triazol-4-yl or tetrazol-1-yl radicals, unsubstituted or C-substituted by phenyl or phenyl which is substituted as indicated or by C₁-C₄ alkyl such as methyl, for example imidazol-1-yl, 2-, 4- or 5-C₁-C₄ alkylimidazol-1-yl such as 2-, 4- or 5-methylimidazol-1-yl, pyrazol-1-yl, 3- or 4-C₁-C₄ alkylpyrazol-1-yl such as 3- or 4-methylpyrazol-1-yl, 1H-1,2,4-tetrazol-1-yl, 3-C₁-C₄ alkyl-1H-1,2,4-triazol-1-yl such as 3-methyl-1H-1,2,4-triazol-1-yl, 4H-1,2,4-triazol-1-yl, 3-C₁-C₄ alkyl-4H-1,2,4-triazol-4-yl such as 3-methyl-4H-1,2,4-triazol-4-yl or 1H-1,2,4-tetrazol-1-yl.

Radicals and compounds hereinafter qualified by the term “lower” will be understood as meaning typically those containing up to 7 carbon atoms inclusive, preferably up to 4 carbon atoms inclusive. The general terms have for example the following meanings:

Lower alkyl is for example C₁-C₄ alkyl such as methyl, ethyl, propyl or butyl, and also isobutyl, sec-butyl or tert-butyl, and may further be C₅-C₇ alkyl such as pentyl, hexyl or heptyl.

Phenyl-lower alkyl is for example phenyl-C₁-C₄ alkyl, preferably 1-phenyl-C₁-C₄ alkyl such as benzyl.

Lower alkoxy is for example C₁-C₄ alkoxy such as methoxy, ethoxy, propoxy, isopropoxy, butoxy, isobutoxy, sec-butoxy or tert-butoxy.

Di-lower alkylamino is for example di-C₁-C₄ alkylamino such as dimethylamino, diethylamino, N-ethyl-N-methylamino, dipropylamino, N-methyl-N-propylamino or dibutylamino.

Lower alkylthio is for example C₁-C₄ alkylthio such as methylthio, ethylthio, propylthio or butylthio, and also isobutylthio, sec-butylthio or tert-butylthio.

Halogen is for example halogen having an atomic number of up to 35 inclusive, such as fluorine, chlorine or bromine.

Salts of compounds of formula I are in particular the salts thereof with pharmaceutically acceptable bases, such as non-toxic metal salts derived from metals of groups Ia, Ib, IIa and IIb, e.g. alkali metal salts, preferably sodium or potassium salts, alkaline earth metal salts, preferably calcium or magnesium salts, copper, aluminum or zinc salts, and also ammonium salts with ammonia or organic amines or quaternary ammonium bases such as free or C-hydroxylated aliphatic amines, preferably mono-, di- or tri-lower alkylamines, e.g. methylamine, ethylamine, dimethylamine or diethylamine, mono-, di- or tri(hydroxy-lower alkyl)amines such as ethanolamine, diethanolamine or triethanolamine, tris(hydroxymethyl)aminomethane or 2-hydroxy-tert-butylamine, or N-(hydroxy-lower alkyl)-N,N-di-lower alkylamines or N-(polyhydroxy-lower alkyl)-N-lower alkylamines such as 2-(dimethylamino)ethanol or D-glucamine, or quaternary aliphatic ammonium hydroxides, e.g. with tetrabutylammonium hydroxide.

In this connection it should also be mentioned that the compounds of formula I may also be obtained in the form of inner salts, provided the group R1 is sufficiently basic. These compounds can therefore also be converted into the corresponding acid addition salts by treatment with a strong protic acid such as a hydrohalic acid, sulfuric acid, sulfonic acid, e.g. methanesulfonic acid or p-toluenesulfonic acid, or sulfamic acid, e.g. N-cyclohexylsulfamic acid.

In one embodiment, the therapeutic agents are compounds of formula I, wherein R1 is an imidazolyl, pyrazolyl, 2H-1,2,3-triazolyl, 1H-1,2,4-triazolyl or 4H-1,2,4-triazolyl, tetrazolyl, oxazolyl, isoxazolyl, oxadiazolyl, thiazolyl or thiadiazolyl radical which is unsubstituted or C-substituted by one or two members selected from lower alkyl, lower alkoxy, phenyl or phenyl which is in turn substituted by one or two members selected from lower alkyl, lower alkoxy and/or halogen, hydroxy, di-lower alkylamino, lower alkylthio and/or halogen, and/or is N-substituted at a N-atom which is capable of substitution by lower alkyl or phenyl-lower alkyl which is unsubstituted or substituted by one or two members selected from lower alkyl, lower alkoxy and/or halogen; and R2 is hydrogen, hydroxy, amino, lower alkylthio or halogen, and salts thereof, especially the inner salts and pharmaceutically acceptable salts thereof with bases.

In one embodiment, the therapeutic agents are compounds of formula I, wherein R1 is an imidazolyl, pyrazolyl, 2H-1,2,3-triazolyl or 4H-1,2,4-triazolyl, tetrazolyl, oxazolyl, isoxazolyl, oxadiazolyl, thiazolyl or thiadiazolyl radical which is unsubstituted or C-substituted by one or two members selected from lower alkyl, lower alkoxy, phenyl or phenyl which is in turn substituted by one or two members selected from lower alkyl, lower alkoxy and/or halogen, hydroxy, di-lower alkylamino, lower alkylthio and/or halogen, and/or is N-substituted at a N-atom which is capable of substitution by lower alkyl or phenyl-lower alkyl which is unsubstituted or substituted by one or two members selected from lower alkyl, lower alkoxy and/or halogen; and R2 is hydrogen, hydroxy, amino, lower alkylthio or halogen, and salts thereof, especially the inner salts and pharmaceutically acceptable salts thereof with bases.

In one embodiment, the therapeutic agents are compounds of formula I, wherein R1 is an imidazolyl radical, such as imidazol-1-yl, imidazol-2-yl or imidazol-4-yl, a 4H-1,2,4-triazolyl radical such as 4H-1,2,4-triazol-4-yl, or a thiazolyl radical such as thiazol-2-yl, which radical is unsubstituted or C-substituted by one or two members selected from C.sub.1-C.sub.4 alkyl such as methyl, C₁-C₄ alkoxy such as methoxy, phenyl, hydroxy, di-C₁-C₄ alkylamino such as dimethylamino or diethylamino, C₁-C₄ alkylthio such as methylthio, and/or halogen having an atomic number up to 35 inclusive such as chlorine, and/or is N-substituted at a N-atom which is capable of substitution by C₁-C₄ alkyl such as methyl, or phenyl-C₁-C₄ alkyl such as benzyl; and R2 is preferably hydroxy or, less preferably, hydrogen or amino, and salts thereof, especially the inner salts and pharmaceutically acceptable salts thereof with bases.

In one embodiment, the therapeutic agents are compounds of formula I, wherein R1 is an imidazol-2- or -4-yl radical which is unsubstituted or C-substituted by phenyl or C- or N-substituted by C₁-C₄ alkyl such as methyl, e.g. imidazol-2-yl, 1-C₁-C₄ alkylimidazol-2-yl such as 1-methylimidazol-2-yl, or 2- or 5-C₁-C₄ alkylimidazol-4-yl such as 2- or 5-methylimidazol-4-yl, or is an unsubstituted thiazolyl radical, e.g. thiazol-2-yl, or is a 1H-1,2,4-triazolyl radical which is unsubstituted or substituted by C₁-C₄ alkyl such as methyl, e.g. 1C₁-C₄ alkyl-1H-1,2,4-triazol-5-yl such as 1-methyl-1H-1,2,4-triazol-5-yl, and R2 is hydroxy or, less preferably, hydrogen, and salts, especially pharmaceutically acceptable salts, thereof.

In one embodiment, the therapeutic agents are compounds of formula I, wherein R1 is an imidazol-1-yl, pyrazol-1-yl, 1H-1,2,4-triazol-1-yl, 4H-1,2,4-triazol-4-yl or tetrazol-1-yl radical which is unsubstituted or C-substituted by phenyl or C₁-C₄ alkyl such as methyl, e.g. imidazol-1-yl, 2-, 4- or 5-C₁-C₄ alkylimidazol-1-yl such as 2-, 4- or 5-methylimidazol-1-yl, pyrazol-1-yl, 3- or 4-C₁-C₄ alkylpyrazol-1-yl such as 3- or 4-methylpyrazol-1-yl, 1H-1,2,4-tetrazol-1-yl, 3-C₁-C₄ alkyl-1H-1,2,4-triazol-1-yl such as 3-methyl-1H-1,2,4-triazol-1-yl, 4H-1,2,4-triazol-1-yl, 3-C₁-C₄ alkyl-4H-1,2,4-triazol-4-yl such as 3-methyl-4H-1,2,4-triazol-4-yl or 1H-tetrazol-1-yl, and R2 is hydroxy or, less preferably, hydrogen, and salts, especially pharmaceutically acceptable salts, thereof.

In one embodiment, the therapeutic agents are compounds of formula I, wherein R1 is an imidazolyl radical which is unsubstituted or substituted by C₁-C₄ alkyl such as methyl, e.g. imidazol-1-yl, imidazol-2-yl, 1-methylimidazol-2-yl, imidazol-4-yl or 2- or 5-methylimidazol-4-yl, and R2 is hydroxy or, less preferably, hydrogen, and salts, especially pharmaceutically acceptable salts, thereof.

In a preferred embodiment of the invention, the liposomes contain an entrapped drug for treatment of a solid tumor, such as zoledronic acid.

The outer surface of the liposome may contain a surface coating of hydrophilic polymers comprised of hydrophilic polymer chains, which are preferably densely packed to form a brushlike coating effective to shield liposome surface components. According to the invention, the hydrophilic polymer chains are connected to the liposome lipids chemically.

The outer surface of liposome may contain affinity moieties, effective to bind specifically to a target, e.g., a biological surface such as a cell membrane, a cell matrix, a tissue or target surface or region at which the liposome-based therapy is aimed. The affinity moiety is bound to the outer liposome surface by covalent attachment to surface lipid components and/or to the hydrophilic polymer coat in the liposomes. The affinity moiety is a ligand effective to bind specifically and with high affinity to ligand-binding molecules carried on the target. For example, in one embodiment, the affinity moiety is effective to bind to a tumor-specific antigen and/or receptors over expressed in a solid tumor and in another embodiment, the affinity moiety is effective to bind to cells at a site of inflammation. In another embodiment, the affinity moiety is a vitamin, polypeptide or polysaccharide or protein effector.

The liposome of the present invention are for use in administering a therapeutic agent to a target. The therapeutic agent is entrapped within the liposome.

The liposome composition of the present invention is composed primarily of vesicle-forming lipids. Such a vesicle-forming lipid is one which (a) can form spontaneously into bilayer vesicles in water, as exemplified by the phospholipids, or (b) is stably incorporated into lipid bilayers, with its hydrophobic moiety in contact with the interior, hydrophobic region of the bilayer membrane, and its head group moiety oriented toward the exterior and interior, polar surface of the vesicle.

The vesicle-forming lipids of this type are preferably ones having two hydrocarbon chains, typically acyl chains, and a head group, either polar or nonpolar. However, other phospholipids containing four hydrocarbon chains, such as, tetramyristylcardiolipin are also suitable. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids and sterols such as cholesterol or cholesterol derivatives.

Preferred diacyl-chain lipids for use in the present invention include diacyl glycerol, such as, phosphatidylcholine (PC), phosphatidyl ethanolamine (PE), phosphatidylglycerol (PG), phosphatidylserine (PS), phosphatidic acid (PA), phosphatidylinositol (PI), sphingomyelin (SPM), cardiolipin and the like, alone or in combination. These lipids are preferred for use as the vesicle-forming lipid, the major liposome component, and for use in the derivatized lipid described below.

Additionally, the vesicle-forming lipid is selected to achieve a specified degree of fluidity or rigidity, to control the stability of the liposome in serum and to control the rate of release of the entrapped agent in the liposome. The rigidity of the liposome, as determined by the vesicle-forming lipid, may also play a role in fusion of the liposome to a target cell, as will be described.

Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures.

The liposomes of the invention may contain a hydrophilic polymer coating made up of polymer chains which are linked to liposome surface lipid. Such hydrophilic polymer chains are incorporated in the liposome by including between about 1-20 mole percent hydrophilic polymer-lipid conjugate. Hydrophilic polymers suitable for use in the polymer coating include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyglycerine and polyaspartamide, hyaluronic acid.

In a preferred embodiment, the hydrophilic polymer is polyethyleneglycol (PEG), preferably as a PEG chain having a molecular weight between 500-10,000 daltons, more preferably between 2,000-10,000 daltons and most preferably between 1,000-5,000 daltons.

In another preferred embodiment, the hydrophilic polymer is polyglycerine (PG), preferably as a PG chain having a molecular weight between 400-2000 daltons, more preferably between 500-1,000 daltons and most preferably between 600-700 daltons.

The liposome composition of the present invention may contain an affinity moiety. The affinity moiety is generally effective to bind specifically to a target, that is, a biological surface such as a target cell surface or membrane, cell surface receptors, a cell matrix, a region of plaque, or the like. The affinity moieties are bound to the liposome surface by direct attachment to a liposomal lipid either to a phospholipid or to cholesterol or by attachment through a short polymer chain, as will be described.

In one embodiment, the affinity moiety is a ligand effective to bind specifically with a receptor at the target region, more specifically, a ligand for binding to a receptor on a target cell. Non-limiting examples of ligands suitable for this purpose are listed in Table 1.

TABLE 1
LIGAND-RECEPTOR PAIRS
AND ASSOCIATED TARGET CELL

| | | Epithelial carcinomas, |
|---|---|---|
| Folate | Folate receptor | bone marrow stem cells |
| Water soluble vitamins | Vitamin receptor | Various cells |
| Pyridoxyl phosphate | CD4 | CD4+ lymphocytes |
| Apolipoproteins | LDL | Liver hepatocytes, |
| | | Vascular endothelial cells |
| Insulin | Insulin receptor | |
| Transferrin | Transferring receptor | Endothelial cells |
| | | (brain) |
| Galactose | Asialoglycoprotein | Liver hepatocytes |
| Sialyl-Lewis* | E,P selectin | Activated endothelial |
| | | cells |
| VEGF | Flk-1,2 | Tumor epithelial cells |
| Basic FGF | FGF receptor | Tumor epithelial cells |
| EGF | EGF receptor | Epithelial cells |
| VCAM-1 | A₄β₂-integrin | Vascular endothelial cells |
| ICAM-1 | α_{L}β₂-integrin | Vascular endothelial cells |
| PECAM-1/CD31 | αᵥβ₃-integrin | Vascular endothelial cells |
| Fibronectin | αᵥβ₃-integrin | Activated platelets |
| Osteopontin | αᵥβ₁ and αᵥβ₅-integrin | Smooth muscle cells in |
| | | atherosclerotic plaques |
| RGD sequences of | αᵥβ₃-integrin | Tumor endothelial cells, |
| matrix proteins | | vascular smooth muscle |
| | | cells |

The ligands listed in Table 1 may be used, in one embodiment of the invention, to target the liposomes, to specific target cells. For example, a folate ligand attached to the head group of DSPE or to the distal end of a short PEG chain derivatized to DSPE can be incorporated into the liposomes. A “short” PEG chain, as used herein is meant to specify a PEG chain having a length (molecular weight) selected such that the ligand, when incorporated into the liposome, is masked or shielded by the surface coating of hydrophilic polymer chains. A surface-bound folate ligand incorporated onto the liposome is effective to bind to folate receptors on epithelial cells for administration of an entrapped therapeutic agent to the target cell, for example, administration of a neoplastic agent for treatment of epithelial carcinomas.

The affinity moiety is a short peptide that has cell-binding activity and is effective to compete with a ligand for a receptor site. Inhibition of the ligand-receptor cell-binding event results in arresting an infection process.

Lipid vesicles containing the entrapped agent are prepared according to well-known methods, such as those described above, typically, hydration of a lipid film, reverse-phase evaporation, solvent dilution, detergent dialysis, freeze and thaw and microencapsulation. The compound to be delivered is either included in the organic medium, in the case of a lipophilic compound, or is included in the hydration medium, in the case of a water-soluble therapeutic agent. Alternatively, the therapeutic agent may be loaded into preformed vesicles prior to administration to the subjects.

### II. Liposome Preparation

### A. Preparation of Releasable Polymer Coating

The hydrophilic polymer chains are attached to the liposome through a linkage, that may cleave in response to a selected stimulus. In one embodiment, the linkage is a peptide, ester or disulfide linkage.

A peptide-linked compound is prepared, for example, by coupling a polyalkylether, such as PEG, to a lipid amine. End-capped PEG is activated with a carbonyl diimidazole coupling reagent, to form the activated imidazole compound. The activated PEG is then coupled to with the N-terminal amine of the exemplary tripeptide shown. The peptide carboxyl group can then be used to couple a lipid amine group, through a conventional carbodiimide coupling reagent, such as dicyclohexylcarbodiimide (DCC).

The ester linked compound can be prepared, for example, by coupling a lipid acid, such as phosphatidic acid, to the terminal alcohol group of a polyalkylether, using alcohol via an anhydride coupling agent. Alternatively, a short linkage fragment containing an internal ester bond and suitable end groups, such as primary amine groups, can be used to couple the polyalkylether to the vesicle-forming lipid through amide or carbamate linkages.

### B. Attachment of Affinity Moiety

As described above, the liposomes of the present invention may contain an affinity moiety attached to the surface of the PEG-coated liposomes. The affinity moiety is attached to the liposomes by direct attachment to liposome lipid surface components or through a short spacer arm or tether, depending on the nature of the moiety.

A variety of methods are available for attaching molecules, e.g., affinity moieties, to the surface of lipid vesicles. In one preferred method, the affinity moiety is coupled to the lipid, by a coupling reaction described below, to form an affinity moiety-lipid conjugate. This conjugate is added to a solution of lipids for formation of liposomes. In another method, a vesicle-forming lipid activated for covalent attachment of an affinity moiety is incorporated into liposomes.

In general, attachment of a moiety to a spacer arm can be accomplished by derivatizing the vesicle-forming lipid, typically DSPE, with a hydrophilic polymer, such as PEG, having a reactive terminal group for attachment of an affinity moiety. Methods for attachment of ligands to activated PEG chains are described in the art (Allen, et al., 1995; Zalipsky, 1993; Zalipsky, 1994; Zalipsky, 1995a; Zalipsky, 1995b). In these methods, the inert terminal methoxy group of mPEG is replaced with a reactive functionality suitable for conjugation reactions, such as an amino or hydrazide group. The end functionalized PEG is attached to a lipid, typically DSPE. The functionalized PEG-DSPE derivatives are employed in liposome formation and the desired ligand is attached to the reactive end of the PEG chain before or after liposome formation.
The attachment of a moiety can also be accomplished by derivatizing the cholesterol with a hydrophilic polymer, such as PEG, having a reactive terminal group for attachment of an affinity moiety. Method for attachment of ligands to activated PEG chains are described in the art (Guo, W., Lee, T., Sudimack, J., and Lee, R. J. Receptor-Targeted Delivery of Liposomes via Folate-PEG-Chol, (2000) J. Liposome Res., 10:179-195).

### C. Liposome Preparation

The liposomes may be prepared by a variety of techniques, such as those detailed in Szoka, et al., 1980. Multilamellar vesicles (MLVs) can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

The lipid components used in forming the fusogenic liposomes of the present invention are preferably present in a molar ratio of about 70-95 percent vesicle-forming lipids, 1-20 percent of a lipid derivatized with a hydrophilic polymer chain, and 0.1-5 percent of a lipid having an attached affinity moiety. One exemplary formulation includes 80-95 mole percent phosphatidylcholine, 1-20 mole percent of PEG-DTP-DSPE, and 0.1-5 mole percent of affinity moiety-DSPE. Cholesterol may be included in the formulation at between about 1-50 mole percent.

Another procedure suitable for preparation of the fusogenic liposomes of the present invention is described by Uster, et al., 1996. In this method, liposomes with an entrapped therapeutic agent are prepared from vesicle-forming lipids. The preformed liposomes are added to a solution containing a concentrated dispersion of micelles of affinity moiety-DSPE conjugates and/or PEG-derivatized lipid conjugates and incubated under conditions effective to achieve insertion of the micellular lipid conjugates into the preformed liposomes.

Still another liposome preparation procedure suitable for preparation of the liposomes of the present invention is a solvent injection method. In this procedure, a mixture of the lipids, dissolved in a solvent, preferably ethanol or DMSO, is injected into an aqueous medium with stirring to form liposomes. The solvent is removed by a suitable technique, such as dialysis or evaporation, and the liposomes are then sized as desired. This method achieves relatively high encapsulation efficiencies.

A hydrophilic therapeutic agent is entrapped in the liposomes by including the agent in the aqueous hydration mixture. A hydrophobic therapeutic agent is entrapped in the liposomes by including the agent with the lipids prior to formation of a thin film or dissolved in a lipid solvent prior to injection into an aqueous medium.

The liposomes are preferably prepared to have substantially homogeneous sizes in a selected size range, typically between about 10 to about 500 nm, preferably 50 to about 300 nm and most preferably 80 to about 200 nm.

When desired, the liposomes can be dried such as by evaporation or lyophilization and resuspended in any desirable solvent. Where liposomes are lyophilized, nonreducing sugars can be added prior to lyophilization or during liposome formulation to provide stability. One such sugars is sucrose.

The liposome having a divalent cation matrix can be made by an addition of a solvent containing a divalent cation during liposome preparation.

The liposome having a dilvalent cation matrix can also be made by reconstituted the lyophilized liposomes with a suitable solvent containing a divalent cation prior to administration to the subjects.

It has been found that invented liposomes having a concentration gradient across their membranes can be dehydrated in the presence of one or more sugars, stored in their dehydrated condition, subsequently rehydrated, and the concentration gradient then used to create a transmembrane potential which will load divalent cations into the liposomes and form drug-divalent cation matrix.

When the dehydrated liposomes are to be used, rehydration is accomplished by simply adding an aqueous solution of divalent cations, e.g., calcium chloride, buffer solution containing divalent cations to the liposomes and allowing them to rehydrate and form drug-divalent cation matrix. The liposomes can be resuspended into the aqueous solution by gentle swirling of the solution. The rehydration can be performed at room temperature or at other temperatures appropriate to the composition of the liposomes and their internal contents.

### III. Method of Treatment

The invention includes, in one aspect, a method of liposome-based therapy for a mammalian subject which includes systemically administering to the subject, liposomes containing (i) a divalent cation matrix and (ii) a therapeutic agent. The divalent cation matrix provides protection of a therapeutic agent which otherwise might leak out of traditional liposomal formulation on the shelf and once introduced into the body. Another aspect, the invention includes a method of liposome-based therapy for a mammalian subject which includes systemically administering to the subject liposomes containing (i) a divalent cation matrix, (ii) a therapeutic agent, (iii) a hydrophilic polymer coating for stability and prolonged circulation; and (iv) optionally an affinity moiety effective to bind specifically to a target surface at which the therapy is aimed The hydrophilic polymer coating is made up of polymer chains which are covalently linked to surface lipid components in the liposomes. The administered liposomes are allowed to circulate systemically until a desired biodistribution of the liposomes is achieved, thereby to expose the affinity agent to the target surface.

In a preferred embodiment, the liposomes are used for treatment of a solid tumor. The liposomes include an anti-tumor drug in entrapped form and are targeted to the tumor region by an affinity moiety effective to bind specifically to a tumor-specific antigen. For example, liposomes can be targeted to the vascular endothelial cells of tumors by including a VEGF ligand in the liposome, for selective attachment to Flk-1,2 receptors expressed on the proliferating tumor endothelial cells.

In this embodiment, the liposomes are sized to between about 10-200 nm, preferably 50-150 nm and most preferably 80-120 nm. Liposomes in this size range have been shown to be able to enter tumors through “gaps” present in the endothelial cell lining of tumor vasculature (Yuan, et al., 1995).

In one embodiment the therapeutic agents are selected from the compounds of formula I. The compounds of formula I and salts thereof have valuable pharmacological properties. In particular, they have a pronounced regulatory action on the calcium metabolism of warm-blooded animals. Most particularly, they effect a marked inhibition of bone resorption in rats, as can be demonstrated in the experimental procedure described in Acta Endrocinol. 78, 613-24 (1975), by means of the PTH-induced increase in the serum calcium level after subcutaneous administration of doses in the range from about 0.01 to 1.0 mg/kg, as well as in the TPTX (thyroparathyroidectomised) rat model by means of hypercalcaemia induced by vitamin D.sub.3 after subcutaneous administration of a dose of about 0.0003 to 1.0 mg. Tumor calcaemia induced by Walker 256 tumors is likewise inhibited after peroral administration of about 1.0 to 100 mg/kg. In addition, when administered subcutaneously in a dosage of about 0.001 to 1.0 mg/kg in the experimental procedure according to Newbould, Brit. J. Pharmacology 21, 127 (1963), and according to Kaibara et al., J. Exp. Med. 159, 1388-96 (1984), the compounds of formula I and salts thereof effect a marked inhibition of the progression of arthritic conditions in rats with adjuvant arthritis. They are therefore eminently suitable for use as medicaments for the treatment of diseases which are associated with impairment of calcium metabolism, for example inflammatory conditions in joints, degenerative processes in articular cartilege, of osteoporosis, periodontitis, hyperparathyroidism, and of calcium deposits in blood vessels or prothetic implants. Favorable results are also achieved in the treatment of diseases in which an abnormal deposit of poorly soluble calcium salts is observed, as in arthritic diseases, e.g. ancylosing spondilitis, neuritis, bursitis, periodontitis and tendinitis, fibrodysplasia, osteoarthrosis or arteriosclerosis, as well as those in which an abnormal decomposition of hard body tissue is the principal symptom, e.g. heriditary hypophosphatasia, degenerative states of articular cartilege, osteoporosis of different provenance, Paget's disease and osteodystrophia fibrosa, and also osteolytic conditions induced by tumors.

After administration of the liposomes, e.g., intravenous administration, and after sufficient time has elapsed to allow the liposomes to distribute through the subject and extravasate into the tumor, the affinity moiety of the liposomes provides binding and internalization into the target cells. In one embodiment, the hydrophilic surface coating is attached to the liposomes by a pH sensitive linkage, and the linkages are released after the liposomes have extravasated into the tumor, due to the hypoxic nature of the tumor region.

From the foregoing, it can be appreciated how various features and objects of the invention are met. The liposomes of the present invention provide a method for targeting liposomes. The hydrophilic surface coating reduces uptake of the liposomes, achieving a long blood circulation lifetime for distribution of the liposomes. After distribution, the liposome-attached affinity moieties allow for multi-valent presentation and binding with the target.

The following examples illustrate methods of preparing, characterizing, and using the liposomes of the present invention. The examples are in no way intended to limit the scope of the invention. Although the invention has been described with respect to particular embodiments, it will be apparent to those skilled in the art that various changes and modifications can be made without departing from the invention.

### EXAMPLE 1

Seven hundred and seventy μmoles of phosphatidyl choline and 330 μmoles cholesterol are dissolved in methylene chloride. The mixture is stirred so that the solvents evaporate under vacuum at about 36° C. to form a thin dry film of lipids. To this mixture, zoledronic acid (110 μmoles) containing 15 ml of sucrose solution is added and vortexed. The unilamellar liposomes are prepared by using a sonicator. The efficiency of drug encapsulation is determined by dialyzing an aliquot of the liposomes overnight in a suitable aqueous solvent or centrifuging an aliquot of the liposomes at 200,000×g. for 2 hours. Thereafter the liposome fraction is dissolved in methanol and analyzed by standard methods using high pressure liquid chromatography (HPLC), such as reverse phase HPLC.

### EXAMPLE 2

The lipids (distearoylphasphatidylcholine, polyglycerine, cholesterol) are dissolved in the methylene chloride. The lipid solution is evaporated using a rotary evaporator under vacuum. After evaporation, the lipid residue is further dried overnight in a dessicator. Zoledronic acid, sucrose and sodium chloride are dissolved in de-ionized water to achieve the required batch concentrations. Then, the dried lipid residue is hydrated in a zoledronic acid, sucrose/NaCl solution to form multi-lamellar vesicles (MLV). The size of the MLV is reduced by extrusion through 0.2 μm, and 0.1 μm polycarbonate filters. Five millimeters of the final formulation is filled into glass vials and freeze-dried using a VIRTIS Lyophilizer. The lyophilized liposomal zoledronic acid is reconstituted with calcium buffer prior to administration to the subject.

### EXAMPLE 3

Cationic phospholipid, DPPC, folate-PEG-DSPE, cholesterol are dissolved in ethanol. The lipid alcohol mixture is then dispersed in Zoledronic acid/sucrose solution. The bulk liposomal zoledronic acid is then extruded through 0.2 μM and 0.1 μM polycarbonate filters. Following size-reduction, the product was then heated to 40° C. under vacuum to evaporate the organic solvent and then sterile filtered through 0.22 μM filters and lyophilized. The drug entrapment efficiency is about 50% assay by HPLC method.

### EXAMPLE 4

DSPC, PEG-cholesterol, folate-PEG-cholesterol are dissolved in ethanol. The lipid alcohol mixture is then dispersed in Zoledronic acid/sucrose solution. The bulk liposomal zoledronic acid is then extruded through 0.2 μM and 0.1 μM polycarbonate filters. Following size-reduction, the product was then heated to 40° C. under vacuum to evaporate the organic solvent and then sterile filtered through 0.22 μM filters and lyophilized.

1: A method of administering a therapeutic agent to a mammalian subject, comprising systemically administering to the subject, liposome composition comprising a divalent cation matrix which contains a therapeutic agent.

2: The method of claim 1, wherein said therapeutic agent is water soluble.

3: The method of claim 2, wherein said therapeutic agent is a compound of formula I: 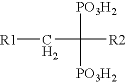
wherein R1 is a 5-membered heteroaryl radical which contains, as hetero atoms, 2 to 4 N-atoms or 1 or 2 N-atoms as well as 1 O- or S-atom, and which is unsubstituted or C-substituted by lower alkyl, phenyl or phenyl which is substituted by lower alkyl, lower alkoxy and/or halogen, or by lower alkoxy, hydroxy, di-lower alkylamino, lower alkylthio and/or halogen, and/or is N-substituted at a N-atom which is capable of substitution by lower alkyl, lower alkoxy and/or halogen, and R2 is hydrogen, hydroxy, amino, lower alkylthio or halogen, and pharmaceutically acceptable salts thereof.

4: The method of claim 3, wherein said therapeutic agent is zoledronic acid.

5: The method of claim 1, wherein the divalent cation matrix comprises divalent cations, such as, calcium ions or Zinc cations or magnesium cations.

6: The method of claim 1, wherein the divalent cation matrix comprises cationic lipids.

7: The method of claim 1, wherein the liposome composition has an average particle size of about 10 to about 500 nanometers.

8: The method of claim 1, wherein the liposome composition further comprises a hydrophilic polymer.

9: The method of claim 1, wherein the liposome composition further comprises an affinity moiety.

10: A method of administering a therapeutic agent to a mammalian subject, comprising systemically administering to the subject, a liposome composition comprising a divalent cation matrix which contains a therapeutic agent.

11: The method of claim 10, for administering a therapeutic agent to target cells, wherein the affinity moiety is a ligand effective to bind specifically with a cell-surface receptor on the target cells, and the liposomes further include the therapeutic agent in entrapped form.

12: The method of claim 10, wherein the affinity moiety is effective to bind specifically to a tumor-specific antigen.

13: The method of claim 10, wherein said therapeutic agent is water soluble.

14: The method of claim 10, wherein said therapeutic agent is a compound of formula I: 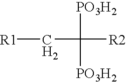
wherein R1 is a 5-membered heteroaryl radical which contains, as hetero atoms, 2 to 4 N-atoms or 1 or 2 N-atoms as well as 1 O- or S-atom, and which is unsubstituted or C-substituted by lower alkyl, phenyl or phenyl which is substituted by lower alkyl, lower alkoxy and/or halogen, or by lower alkoxy, hydroxy, di-lower alkylamino, lower alkylthio and/or halogen, and/or is N-substituted at a N-atom which is capable of substitution by lower alkyl, lower alkoxy and/or halogen, and R2 is hydrogen, hydroxy, amino, lower alkylthio or halogen, and pharmaceutically acceptable salts thereof.

15: The method of claim 10, wherein said therapeutic agent is zoledronic acid.

16: The method of claim 10, wherein the divalent cation matrix comprises divalent cations, such as, calcium ions or Zinc cations or magnesium cations.

17: The method of claim 10, wherein the divalent cation matrix comprises cation lipids.

18: The method of claim 10, wherein the liposome composition has an average particle size of about 10 to about 500 nanometers.

19: The method of claim 10, wherein the liposome composition further comprises a hydrophilic polymer.

20: The method of claim 10, wherein the liposome composition further comprises an affinity moiety.

21: A liposome composition comprising a divalent cation matrix which contains a therapeutic agent.

22: The composition of claim 21, wherein said therapeutic agent is water soluble.

23: The composition of claim 21, wherein said therapeutic agent is a compound of formula I: 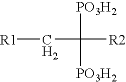
wherein R1 is a 5-membered heteroaryl radical which contains, as hetero atoms, 2 to 4 N-atoms or 1 or 2 N-atoms as well as 1 O- or S-atom, and which is unsubstituted or C-substituted by lower alkyl, phenyl or phenyl which is substituted by lower alkyl, lower alkoxy and/or halogen, or by lower alkoxy, hydroxy, di-lower alkylamino, lower alkylthio and/or halogen, and/or is N-substituted at a N-atom which is capable of substitution by lower alkyl, lower alkoxy and/or halogen, and R2 is hydrogen, hydroxy, amino, lower alkylthio or halogen, and pharmaceutically acceptable salts thereof.

24: The composition of claim 21, wherein said therapeutic agent is zoledronic acid.

25: The composition of claim 21, wherein the divalent cation matrix comprises divalent cations, such as, calcium ions or Zinc cations or magnesium cations.

26: The method of claim 21, wherein the liposome composition further comprises a hydrophilic polymer.

27: The method of claim 21, wherein the liposome composition further comprises an affinity moiety.

28: A liposome composition comprising a (a) therapeutic agent; (b) a divalent cation matrix, (c) a hydrophilic polymer coating; and (d) optionally an affinity moiety.

29: The liposome composition of claim 28, wherein the affinity moiety is a ligand effective to bind specifically with a cell-surface receptor on the target surface.

30: The liposome composition of claim 28, wherein the affinity moiety is effective to bind specifically to a tumor-specific antigen.

31: The liposome composition of claim 28, wherein said therapeutic agent is water soluble.

32: The liposome composition of claim 28, wherein said therapeutic agent is a compound of formula I: 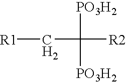
wherein R1 is a 5-membered heteroaryl radical which contains, as hetero atoms, 2 to 4 N-atoms or 1 or 2 N-atoms as well as 1 O- or S-atom, and which is unsubstituted or C-substituted by lower alkyl, phenyl or phenyl which is substituted by lower alkyl, lower alkoxy and/or halogen, or by lower alkoxy, hydroxy, di-lower alkylamino, lower alkylthio and/or halogen, and/or is N-substituted at a N-atom which is capable of substitution by lower alkyl, lower alkoxy and/or halogen, and R2 is hydrogen, hydroxy, amino, lower alkylthio or halogen, and pharmaceutically acceptable salts thereof.

33: The liposome composition of claim 28, wherein said therapeutic agent is zoledronic acid.

34: The liposome composition of claim 28, wherein the divalent cation matrix comprises divalent cations, such as, calcium ions or Zinc cations or magnesium cations.

35: The liposome composition of claim 28, wherein the divalent cation matrix comprises cationic lipids.

36: The liposome composition of claim 28, wherein the liposome composition has an average particle size of about 10 nanometer to about 500 nanometers.
